(19) United States
(12) Patent Application Publication
(10) Pub. No.: US 2024/0130968 A1
(43) Pub. Date: Apr. 25, 2024

(54) METHODS AND PRODUCTS FOR TREATING SUBJECTS WITH AUTISM SPECTRUM DISORDERS

(71) Applicant: NEURIM PHARMACEUTICALS (1991) LTD., Tel Aviv (IL)

(72) Inventors: Nava ZISAPEL, Tel Aviv (IL); Ran FURMAN, Tel Aviv (IL)

(73) Assignee: NEURIM PHARMACEUTICALS (1991) LTD., Tel Aviv (IL)

(21) Appl. No.: 18/390,481

(22) Filed: Dec. 20, 2023

Related U.S. Application Data
(63) Continuation-in-part of application No. 17/937,525, filed on Oct. 3, 2022, now Pat. No. 11,878,016.
(60) Provisional application No. 63/251,935, filed on Oct. 4, 2021.

(51) Int. Cl. A61K 9/107; A61J 7/00; A61K 31/315; A61K 31/519; A61K 45/06; A61P 25/00

(52) U.S. Cl. CPC A61K 9/1075; A61J 7/0084; A61K 31/315; A61K 31/519; A61K 45/06; A61P 25/00

(57) ABSTRACT

Methods and products for treating a subject diagnosed with an autism spectrum disorder, an intellectual disability, an anxiety disorder, a mood disorder, a disorder of social interaction, irritability, aggression, self-injurious behavior, hyperactivity, inattention, or Fragile X syndrome or brain neuroinflammation by administering a water-insoluble ticagrelor or ticagrelor salt, pre-dissolved in oil/surfactant/cosurfactant mixture and emulsified in an aqueous solution, containing a second agent, which may include a magnesium ion containing-compound, a zinc ion containing-compound, a lysine or lysine salt, an arginine or arginine salt, lecithin, or a combination thereof.

### CROSS-REFERENCE TO RELATED APPLICATIONS

This application is a continuation-in-part of U.S. application Ser. No. 17/937,525, filed Oct. 3, 2022, which claims priority to and the benefit of Provisional Patent Application No. 63/251,935, filed on Oct. 4, 2021, the entire contents of which are incorporated herein by reference.

### FIELD OF THE DISCLOSURE

This disclosure relates to methods and compositions for treating autism spectrum disorders and associated disorders.

### BACKGROUND

Autism or autism spectrum disorder (ASD) is a complex neurodevelopmental condition characterized by persistent difficulties in social interactions and communication, as well as restricted interests and repetitive behaviors. ASD is typically diagnosed during the first three years of life and manifests in characteristic symptoms or behavioral traits (core symptoms). A diagnosis of ASD now includes several conditions that used to be diagnosed separately: autistic disorder, pervasive developmental disorder not otherwise specified (PDD-NOS), and Asperger syndrome. All of these conditions are now encompassed by the diagnostic criteria for autism spectrum disorder as set forth in the American Psychiatric Association's Diagnostic & Statistical Manual of Mental Disorders, Fifth Edition (DSM-V).

Despite advances in early diagnosis and behavior intervention, there is no Food and Drug Administration (FDA)-approved medication specifically targeting core symptoms of ASD (social communication deficits and rigid adherence to routines and repetitive behaviors) to date. The mainstays of treatment for autism are behavioral therapy and educational interventions. The pharmaceutical industry has repeatedly failed in developing an effective treatment for autism, with a string of product failures in recent years including oxytocin, arbaclofen, memantine, and mavoglurant. Clinical trials with the breakthrough-tagged Vasopressin 1a Receptor Antagonist balovaptan (RG7314) to improve social communication in Autism and bumetanide (a diuretic agent, that reduces intracellular chloride—thereby reducing neuronal excitability) to improve communicative and cognitive abilities were recently stopped for futility. At present, the only US Food and Drug Administration (FDA) approved drugs for autism are the atypical antipsychotics, which aim to treat the associated symptom of irritability. The top-selling drugs used for the symptomatic treatment of autism are Otsuka Pharmaceutical's Abilify (aripiprazole) and Johnson and Johnson's Risperdal (risperidone).

Multiple studies report that an abnormal immune function, which includes inflammation, cytokine dysregulation, and anti-brain autoantibodies, can significantly influence the development of ASD. Some individuals may not display outwards signs of infection but instead suffer from subclinical (or asymptomatic) infections (Lintas et al. Association of autism with polyomavirus infection in postmortem brains. J Neurovirol. 2010 March; 16(2):141-9).

Children <7 years old as well as older patients (e.g., suffering from dementia) are not always able to swallow solid pharmaceutical forms, and this becomes an intractable hurdle in dosing of children with neurodevelopmental delays. Moreover, drug dosage is determined by the age and weight of each pediatric patient. Active pharmaceutical ingredients with low aqueous solubility may be permeable and well absorbed after oral administration but if they are nearly insoluble in water the orally administered forms are in a solid state and their formulation as an oral liquid solution is challenging. Moreover, formulation of combinations of water-soluble and water-insoluble drugs as an oral liquid solution is challenging. There is a long-felt, but unmet need for pharmaceutical products to treat the core symptoms of autism spectrum disorder and autism.

### SUMMARY OF THE INVENTION

The present disclosure includes liquid, lipid nanoparticle emulsion (LNE) formulation and methods for treating a subject having disorders of social interaction, anxiety, irritability, aggression, tantrums, rapid changes in mood, self-injurious behavior (SIB), hyperactivity, inattention, ASD and neurological disorders (e.g., neurodegenerative, neurogenetic, neurodevelopmental and neuroinflammatory disorders). In certain aspects, the present disclosure includes administering lipid nanocarrier emulsion (LNE) formulation comprising a water-insoluble drug (Ticagrelor) pre-dissolved in an oil/surfactant/cosurfactant mixture and emulsified in an aqueous solution containing the second, water-soluble agent (e.g., zinc salt, magnesium salt, etc.), to the subject.

Ticagrelor is also known as (1S,2S,3R,5S)-3-[7-[[(1R,2S)-2-(3,4-difluorophenyl)cyclopropyl]amino]-5-(propylthio)-3H-1,2,3-triazolo[4,5-d]pyrimidin-3-yl]-5-(2-hydroxyethoxy)-1,2-cyclopentanediol). The present disclosure also includes an enantiomer of ticagrelor or a pharmaceutically acceptable salt thereof. For example, one enantiomer of ticagrelor is (1R,2R,3S,5R)-3-(7-(((1S,2R)-2-(3,4-Difluorophenyl)cyclopropyl)amino)-5-(propylthio)-3H-[1,2,3]triazolo[4,5-d]pyrimidin-3-yl)-5-(2-hydroxyethoxy)cyclopentane-1,2-diol. Unless stated otherwise, the term ticagrelor should be construed broadly to include enantiomers thereof.

In one aspect, the present disclosure includes a method of treating a subject diagnosed with ASD, an intellectual disability, epilepsy, an anxiety disorder, a mood disorder, a disorder of social interaction, irritability, aggression, self-injurious behavior, hyperactivity, inattention, Fragile X syndrome by administering ticagrelor, an enantiomer thereof, or a pharmaceutically acceptable salt thereof and a second agent in LNE formulation to the subject.

In one aspect, the present disclosure includes a method of treating a subject with a neuroinflammatory, neurogenetic, neurodegenerative or neurodevelopmental disorder by administering ticagrelor, an enantiomer thereof, or a pharmaceutically acceptable salt thereof and a second agent in an LNE formulation to the subject.

In one aspect, the formulation includes an oil/surfactant/cosurfactant mixture emulsified in an aqueous solution.

In one aspect, the oil/surfactant/cosurfactant mixture to aqueous solution ratio is 1:0.1 to 1:100 (volume/volume).

In one aspect, the present disclosure includes LNE containing ticagrelor, an enantiomer, or a pharmaceutically acceptable salt thereof and a second agent. The LNEs formulation of the present disclosure are in a liquid forms. A person of ordinary skill in the art will immediately recognize that the ticagrelor in LNE formulation is at a different dosage form than other oral dosage forms, e.g., a chewable tablet or a tablet that should be swallowed whole with liquid, and has different physical and pharmaceutical properties, manufacturing techniques, and regulatory requirements.

In the human body, biological barriers such as the skin, blood-brain barrier (BBB), mucosal membranes and membranes around cells and nuclei, etc., limit the delivery of drugs to the desired sites to achieve the necessary therapeutic actions. Lipid-based nanocarriers may facilitate the penetration of drugs across these obstacles, leading to improvements in their effectiveness. LNEs by virtue of their formulation have specific physical characteristics which increase the solubility and bioavailability of poorly water-soluble drugs.

In one aspect, the present disclosure includes administering ticagrelor, an enantiomer, or a pharmaceutically acceptable salt thereof to a subject in combination with a second agent in an oral liquid form.

In one aspect the water insoluble drug will be pre-dissolved in an oil/surfactant/cosurfactant mixture and then emulsified in an aqueous solution (e.g., water, buffer, acid), containing the second agent, resulting in an LNE drug combination product.

In one aspect, the present disclosure includes administering ticagrelor, an enantiomer, or a pharmaceutically acceptable salt thereof to a subject in combination with a zinc salt. In one aspect, the present disclosure includes administering ticagrelor, an enantiomer, or a pharmaceutically acceptable salt thereof to a subject in combination with a magnesium salt. In one aspect, the present disclosure includes administering ticagrelor, an enantiomer, or a pharmaceutically acceptable salt thereof to a subject in combination with lysine. In one aspect, the present disclosure includes administering ticagrelor or a pharmaceutically acceptable salt thereof to a subject in combination with lecithin. In one aspect, the present disclosure includes administering ticagrelor or a pharmaceutically acceptable salt thereof to a subject in combination with lysine and lecithin. In one aspect, the present disclosure includes administering ticagrelor or a pharmaceutically acceptable salt thereof to a subject in combination with lysine and magnesium salt. In one aspect, the present disclosure includes administering ticagrelor or a pharmaceutically acceptable salt thereof to a subject in combination with lysine and zinc salt. In one aspect, the present disclosure includes administering ticagrelor or a pharmaceutically acceptable salt thereof to a subject in combination with an arginine-containing compound.

In one aspect, the amount by weight of ticagrelor in the LNE of the present disclosure is lower than in conventional ticagrelor products, e.g., less than 90 mg, less than 60 mg, less than 40 mg, less than 35 mg, less than 30 mg, or less than 10 mg. In one aspect, the amount by weight of ticagrelor to be administered per day according to the methods of the present disclosure are less than for conventional ticagrelor regimens, e.g., less than 90 mg/day, less than 60 mg/day, less than 40 mg/day, less than 30 mg/day, less than 20 mg/day, less than 10 mg/day, or less than 0.25-0.5 mg/kg bodyweight two to four times daily as required.

In one aspect, the present disclosure includes administering ticagrelor or a pharmaceutically acceptable salt thereof to a subject in combination with a further component, e.g., an acid that complexes with magnesium or zinc, including but not limited to citric acid, ascorbic acid, threonic acid, tartaric acid, a malic acid, fumaric acid, gluconic acid, succinic acid, lactic acid, glyceric acid, and/or an alpha-hydroxy-carboxylic acid. In some aspects, the second agent should be selected such that its water solubility is sufficiently high so that the metal ions are bioavailable. For example, the water solubility of the magnesium or zinc salt should be at least 0.2, 0.3, 0.4, 0.5 or 0.6 g/100 ml at 25° C. to increase the bioavailability of the magnesium or zinc ions.

In one aspect, the present disclosure provides a method according to the present disclosures in which, before administering, the method includes providing:
a) a first container containing an oil/surfactant/cosurfactant mixture comprising ticagrelor, an enantiomer thereof, or a pharmaceutically acceptable salt thereof dissolved in a mixture comprising an oil, a surfactant and a cosurfactant; and
b) a second container containing an aqueous solution comprising a magnesium ion containing-compound, a zinc ion containing-compound, L-lysine or a salt thereof, L-arginine or a salt thereof, lecithin, or a combination thereof.

In one aspect, the present disclosure provides a method according to the present disclosures in which, before administering, providing a dual chamber cartridge, wherein a first chamber of the dual chamber cartridge contains the oil/surfactant/cosurfactant mixture comprising ticagrelor, an enantiomer thereof, or a pharmaceutically acceptable salt thereof dissolved in a mixture comprising an oil, a surfactant and a cosurfactant, and a second chamber of the dual chamber cartridge contains the aqueous solution comprising a magnesium ion containing-compound, a zinc ion containing-compound, L-lysine or a salt thereof, L-arginine or a salt thereof, lecithin, or a combination thereof, wherein the dual chamber cartridge is configured for mixing contents of the first chamber and the second chamber to form the LNE before the administering step.

In one aspect, the present disclosure provides a kit including:
a) a first container containing an oil/surfactant/cosurfactant mixture comprising ticagrelor, an enantiomer thereof, or a pharmaceutically acceptable salt thereof dissolved in a mixture comprising an oil, a surfactant and a cosurfactant; and
b) a second container containing an aqueous solution comprising a magnesium ion containing-compound, a zinc ion containing-compound, L-lysine or a salt thereof, L-arginine or a salt thereof, lecithin, or a combination thereof.

Any device that can mix the (a) with the (b) prior to administration can be used according to the present disclosure, e.g., an admixture device.

In one aspect, the present disclosure provides a dual chamber cartridge, wherein a first chamber of the dual chamber cartridge contains an oil/surfactant/cosurfactant mixture comprising ticagrelor, an enantiomer thereof, or a pharmaceutically acceptable salt thereof dissolved in a mixture comprising an oil, a surfactant and a cosurfactant, and a second chamber of the dual chamber cartridge contains an aqueous solution comprising a magnesium ion containing-compound, a zinc ion containing-compound, L-lysine or a salt thereof, L-arginine or a salt thereof, lecithin, or a combination thereof, wherein the dual chamber cartridge is configured for mixing contents of the first chamber and the second chamber to form a LNE formulation.

In one aspect, the subject has been diagnosed with ASD.

In one aspect, the subject has been diagnosed with neurological disorder (e.g., neurodegenerative, neurogenetic, neurodevelopmental or neuroinflammatory disorders).

In one aspect, the subject has an intellectual disability, epilepsy, an anxiety disorder, a mood disorder, a disorder of social interaction, irritability, aggression, self-injurious behavior, hyperactivity, and/or inattention. In one aspect, the subject has Fragile X syndrome.

In one aspect, the subject is an infant, child or adolescent and in such, the amount of ticagrelor to be administered is calculated per body weight, for example 0.01-3 mg/kg, 1 to 3 times a day.

In one aspect, the subject has been diagnosed with elevated TNFα or inflammatory cytokines as makers of neuroinflammation in plasma.

In one aspect, the present invention addresses the need for improved ticagrelor dosage forms, particularly low-dosage forms that enable the fast and safe delivery of ticagrelor to brains of subjects suffering from autism spectrum disorders and associated disorders.

These and other aspects of the invention will be apparent upon reference to the following detailed description, claims, aspects, procedures, compounds, and/or compositions and associated background information and references, which are hereby incorporated in their entirety.

### BRIEF DESCRIPTION OF THE DRAWINGS

FIG. 1 shows the results of the three-chamber test for time spent in the social area, the center area, or the non-social area for mice treated with SAL-vehicle, SAL-ticagrelor (30 mg/kg), VPA-vehicle, VPA-ticagrelor (0.3 mg/kg), VPA-ticagrelor (10 mg/kg), and VPA-ticagrelor (30 mg/kg). The bars in FIG. 1 from left to right are in the same order as the labels in the figure legend from top to bottom, i.e., SAL-vehicle is the left-most bar and VPA-ticagrelor (30 mg/kg) is right-most bar.

FIG. 2 shows the effects of Ticagrelor/zinc (Com-A) on neuroinflammation in a mouse model of autism.

FIG. 3 shows the effects of Zinc on ticagrelor (TCG) bioavailability in the plasma and brain of rats.

FIG. 4 shows the bioavailability of ticagrelor (TCG) LNE formulation versus water or PBS formulations.

FIG. 5 shows a schematic representation of the Cartridges Double Chamber or dual chamber cartridge device (based on Rahul G. Ingle, Wei-Jie Fang (2021) Int. J. of Pharm. 597, 15).

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

The singular forms “a,” “an,” and “the” include plural referents unless the context clearly dictates otherwise.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e., not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e., “comprising,” “consisting,” or “consisting essentially”) provides direct support for replacement to any of the other transition term not specifically used. For example, amending a term from “comprising” to “consisting essentially of” would find direct support due to this definition.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

Generally herein, the term “or” includes “and/or.”

As used herein, the term “stable” as to the product is used to mean that the total impurity level of ticagrelor is 0.5% or lower and that the oil and water phases are not separated.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain compositions, elements, excipients, ingredients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of method steps, active agents, kits, or compositions described with respect to a LNE or method of treating a certain subject is intended to and does find direct support for embodiments related to compositions, formulations, and methods described in any other part of this disclosure, even if those method steps, active agents, kits, or compositions are not re-listed in the context or section of that embodiment or aspect.

Compositions of the present invention may be administered using any suitable route of administration for the dosage form including but not limited to orally, parenterally (including subcutaneous, intramuscular, intravenous, transcutaneous, subdermal, and intradermal), transmucosally, by inhalation spray, topically, rectally, patch or micropatch, nasally, buccally, vaginally or via an implanted reservoir. In some embodiments, provided compounds or compositions are administrable intravenously and/or intraperitoneally. Suitable dosage forms include, without limitation, a liquid form, a gel form, a semi-liquid (for example, a liquid, such as a viscous liquid, containing some solid) form, a semi-solid (a solid containing some liquid) form, and/or a solid form, for example. Merely by way of example, a tablet form, a capsule form, a food form, a chewable form, a non-chewable form, a slow- or sustained-release form, a non-slow- or non-sustained-release from (e.g., immediate release form), and/or the like, may be employed. Liquid pharmaceutically acceptable compositions can, for example, be prepared by dissolving or dispersing the compounds of the present disclosure in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

Preferably, the compositions are administered orally, intranasally, buccally, or sublingually. If desired, certain sweetening, flavoring or coloring agents may also be added. In some embodiments, a provided oral formulation is formulated for immediate release or sustained/delayed release. In some embodiments, the composition is suitable for buccal or sublingual administration. A provided compound can also be in micro-encapsulated form.

Pharmaceutically acceptable compositions of this invention may also be administered by nasal aerosol or inhalation.

In some aspects, pharmaceutically acceptable compositions of this invention are formulated for intra-peritoneal administration.

The present disclosure provides an LNE formulation containing, (1) ticagrelor, an enantiomer, or a pharmaceutically acceptable salt thereof dissolved in an oil/surfactant/cosurfactant mixture and (2) a second agent dissolved in an aqueous solution.

In some aspects, the second agent may be a magnesium salt, a zinc salt, lysine, lecithin, or a combination thereof. In some aspects, the LNE formulation may further include another component, including but not limited to citric acid, ascorbic acid, threonic acid, tartaric acid, malic acid, fumaric acid, gluconic acid, succinic acid, lactic acid, glyceric acid, an alpha-hydroxy-carboxylic acid, amino acid and their magnesium or zinc salts.

In some aspects, the LNE formulation contains lysine or a lysine salt as the second agent. In some aspects, the LNE formulation contains lecithin as the second agent. In some aspects, the LNE formulation contains a combination of lysine and lecithin as the second agent.

In some aspects, the LNE formulation contains from 5 mg to 200 mg ticagrelor, an enantiomer, or a pharmaceutically acceptable salt thereof. In some aspects, the LNE formulation contains from 1 mg to 90 mg ticagrelor, an enantiomer, or a pharmaceutically acceptable salt thereof. In some aspects, the LNE formulation contains from 4 mg to 60 mg ticagrelor, an enantiomer, or a pharmaceutically acceptable salt thereof. In some aspects, the LNE formulation contains from 4 mg to 50 mg ticagrelor, an enantiomer, or a pharmaceutically acceptable salt thereof. In some aspects, the LNE formulation contains from 5 mg to 50 mg ticagrelor, an enantiomer, or a pharmaceutically acceptable salt thereof. In some aspects, the LNE formulation contains from 5 mg to 40 mg ticagrelor, an enantiomer, or a pharmaceutically acceptable salt thereof. In some aspects, the LNE formulation contains from 2, 3, 4, 5, 6, 7, 8, 9, 10, 15, 20, 25, 30, 35, 40, 45, 50, 55, 60, or 65 mg ticagrelor, an enantiomer, or a pharmaceutically acceptable salt thereof.

In some aspects, the LNE formulation contains from 1 mg to 300 mg of the second agent. In some aspects, the LNE formulation contains from 2 mg to 250 mg of the second agent. In some aspects, the LNE contains from 3 mg to 225 mg of the second agent. In some aspects, the LNE formulation contains from 4 mg to 200 mg of the second agent. In some aspects, LNE formulation contains from 5 mg to 175 mg of the second agent. In some aspects, the LNE formulation contains from 5 mg to 160 mg of the second agent. In some aspects, the LNE formulation contains from 6 mg to 150 mg of the second agent. In some aspects, the LNE formulation contains from 7 mg to 140 mg of the second agent. In some aspects, the LNE formulation contains from 8 mg to 130 mg of the second agent. In some aspects, the LNE formulation contains from 9 mg to 120 mg of the second agent. In some aspects, the LNE formulation contains from 10 mg to 110 mg of the second agent. In some aspects, the LNE formulation contains from 15 mg to 100 mg of the second agent. In some aspects, the LNE formulation contains from 20 mg to 80 mg of the second agent. In some aspects, the LNE formulation contains from 25 mg to 75 mg of the second agent. In some aspects, the LNE formulation contains from 30 mg to 60 mg of the second agent. In some aspects, the LNE formulation contains from 35 mg to 55 mg of the second agent. In some aspects the LNE formulation contains from 40 mg to 50 mg of the second agent. In some aspects, the LNE formulation may contain 2 mg to 160 mg lysine. In some aspects, the second agents are between 10% to 50% by weight of the total weight of the LNE formulation.

In some aspects, the LNE formulation contains ticagrelor or a pharmaceutically acceptable salt thereof and a second agent in a ratio from 1:0.1 to 1:100 (mol/mol). In some aspects, the LNE formulation contains ticagrelor or a pharmaceutically acceptable salt thereof in a ratio of from 1:0.3 to 1:80 (mol/mol) to the second agent. In some aspects, the LNE formulation contains ticagrelor or a pharmaceutically acceptable salt thereof in a ratio of from 1:0.5 to 1:70 (mol/mol) to the second agent. In some aspects the LNE formulation contains ticagrelor or a pharmaceutically acceptable salt thereof in a ratio of from 1:0.8 to 1:60 (mol/mol) to the second agent. In some aspects, the LNE formulation contains ticagrelor or a pharmaceutically acceptable salt thereof in a ratio of from 1:1 to 1:50 (mol/mol) to the second agent. In some aspects, the LNE formulation contains ticagrelor or a pharmaceutically acceptable salt thereof in a ratio of from 1:1 to 1:40 (mol/mol) to the second agent. In some aspects, the LNE formulation contains ticagrelor or a pharmaceutically acceptable salt thereof in a ratio of from 1:1 to 1:30 (mol/mol) to the second agent. In some aspects, the LNE formulation contains ticagrelor or a pharmaceutically acceptable salt thereof in a ratio of from 1:1 to 1:20 (mol/mol) to the second agent. In some aspects, the LNE formulation contains ticagrelor or a pharmaceutically acceptable salt thereof in a ratio of from 1:1 to 1:10 (mol/mol) to the second agent. In some aspects, the LNE formulation contains ticagrelor or a pharmaceutically acceptable salt thereof in a ratio of from 1:1 to 1:9 (mol/mol) to the second agent. In some aspects, the LNE formulation contains ticagrelor or a pharmaceutically acceptable salt thereof in a ratio of from 1:1 to 1:8 (mol/mol) to the second agent. In some aspects, the LNE formulation contains ticagrelor or a pharmaceutically acceptable salt thereof in a ratio of from 1:1 to 1:7 (mol/mol) to the second agent. In some aspects, the LNE formulation contains ticagrelor or a pharmaceutically acceptable salt thereof in a ratio of from 1:1 to 1:6 (mol/mol) to the second agent. In some aspects, the LNE formulation contains ticagrelor or a pharmaceutically acceptable salt thereof in a ratio of from 1:1 to 1:5 (mol/mol) to the second agent. In some aspects, the LNE formulation contains ticagrelor or a pharmaceutically acceptable salt thereof in a ratio of from 1:1 to 1:4 (mol/mol) to the second agent. In some aspects, the LNE formulation contains ticagrelor or a pharmaceutically acceptable salt thereof in a ratio of from 1:1 to 1:3 (mol/mol) to the second agent. In some aspects, the LNE formulation contains ticagrelor or a pharmaceutically acceptable salt thereof in a ratio of from 1:1 to 1:2 (mol/mol) to the second agent. In certain aspects the LNE formulation may contain ticagrelor or a pharmaceutically acceptable salt thereof in a ratio of from 1:0.1 to 1:0.9 (mol/mol) to the second agent.

In some aspects, the LNE formulation contains an oil/surfactant/cosurfactant solution to aqueous solution ratio of 1:100 (v/v). In some aspects, the LNE formulation contains an oil/surfactant/cosurfactant solution to aqueous solution ratio of 1:50 (v/v). In some aspects, the LNE formulation contains an oil/surfactant/cosurfactant solution to water-based solution ratio of 1:20 (v/v). In some aspects, the LNE formulation contains an oil/surfactant/cosurfactant solution to aqueous solution ratio of 1:10 (v/v). In some aspects, the LNE formulation contains an oil/surfactant/cosurfactant solution to water based-solution ratio of 1:5 (v/v). In some aspects, the LNE formulation contains an oil/surfactant/cosurfactant solution to aqueous solution ratio of 1:2 (v/v). In some aspects, the LNE formulation contains an oil/surfactant/cosurfactant solution to aqueous solution ratio of 1:1 (v/v). In some aspects the LNE formulation contains an oil/surfactant/cosurfactant solution to water-based solution ratio of 1:0.5 (v/v). In some aspects, the LNE formulation contains an oil/surfactant/cosurfactant solution to aqueous solution ratio of 1:0.1 (v/v).

In some aspects, the LNE formulation contains ticagrelor as a free base or an acid addition salt of ticagrelor. In some aspects, acid addition salt of ticagrelor is any pharmaceutically acceptable acid addition salt. In some aspects, acid addition salt of ticagrelor includes, for example, ticagrelor hydrochloride, ticagrelor tartrate, ticagrelor threonate, ticagrelor glycerate, ticagrelor citrate, or ticagrelor fumarate.

In some aspects the water-insoluble or partially soluble drug to be pre-dissolved in oil/surfactant/cosurfactant mixture includes medicament for the treatment of ASD, Attention Deficit/Hyperactivity Disorder (ADHD) and attention deficit disorder (ADD) (e.g., Risperidone, Aripiprazole, Modafinil and Guanfacine). In some aspects the water-insoluble or partially-soluble drugs to be pre-dissolved in oil/surfactant/cosurfactant mixture includes medicament for the treatment of neurological, neurodevelopmental, neurodegenerative and neurogenetic disorders (e.g., Perampanel, α-pinene, Huperzine A, Ferulic acid, Oxyresveratrol, Lamotrigine, Stiripentol, Ziprasidone, Olanzapine, Carbamazepine, Zolmitriptan, Ginsenoside, Chlorpromazine, Levosulpiride, haloperidol, Levodopa, Berberine, Curcumin, Bilobalide, Biflavonoid, Ginsenoside Rg1, Genistein, Glabridin, taurursodiol, edaravone, riluzole, Dimethyl fumarate, Ibudilast, Lemborexant, Mirtazapine, Naltrexone, Paliperidone, Quetiapine, Tolcapone, ibiglustat etc.).

In some aspects, the LNE formulation contains one or more magnesium salts or magnesium compounds or zinc salts or zinc compounds. In some aspects, the LNE formulation contains a bromide, an iodide, or a complex of magnesium or zinc compounds with hydrochloric acid, pyrophosphoric acid, tauric acid, sulfuric acid, acetic acid, carbonic acid, citric acid, ascorbic acid, threonic acid, tartaric acid, a malic acid, fumaric acid, gluconic acid, succinic acid, lactic acid, a glyceric acid, a glyceraldehyde, an alpha-hydroxy-carboxylic acid, amino acid and their magnesium or zinc salts. In some aspects, the second agent or salt is not a threonate-containing compound (e.g., not a magnesium threonate). In some aspects, the second agent or salt is not a stearate-containing compound (e.g., not a magnesium stearate).

Formulations of the present disclosure must be palatable, e.g., have acceptable organoleptic properties such as good taste and mouthfeel to maintain patient compliance or adherence to the dosing regimen, while also providing the disclosed pharmacokinetic and bioavailability characteristics to provide the desired therapeutic effect. Taste-masking components can inhibit or delay drug release and decrease drug solubility, thereby providing unacceptable pharmacokinetic properties. Conversely, components of the formulation that increase drug solubility and bioavailability may result in undesirable taste or mouthfeel properties. Accordingly, an acceptable LNE formulation must balance these contradictory characteristics in order to provide a palatable (e.g., taste-masked) composition with acceptable pharmacokinetics.

In some aspects, the active ingredients are encapsulated, e.g., in a matrix, layer, polymer, gel-forming region, and/or gel. Stabilization may be provided such that impurity levels when measured after placing the LNE formulation under accelerated storage conditions at 40° C. and 75% relative humidity for 1-3 months are below requisite pharmaceutically acceptable specifications thereby being suitable for commercial use

In some aspects, the LNE formulation of the present disclosure is terminally sterilized.

A pharmaceutical composition may be comprised of an orally administered liquid formulation, including a drug pre-dissolved in oil/surfactant/cosurfactant mixture for increasing the bioavailability of poorly water-soluble drugs or pharmaceutical compositions. The oil/surfactant/cosurfactant mixture component is comprised of an oil or lipid material, a surfactant, and a cosurfactant. A poorly water-soluble drug or pharmaceutical, like ticagrelor, can be treated according to the present invention and can then be used in combination with other drugs and/or pharmaceutical ingredients which may or may not be poorly water-soluble.

The oil/surfactant/cosurfactant mixture of the invention may be formulated for oral administration of ticagrelor, e.g., emulsions, aqueous or oily suspensions or syrups.

The oil phase of the oil/surfactant/cosurfactant mixture includes one or more lipids or glycerides containing compounds such as caprylic/capric acid glycerides but can also include other suitable oil phase compounds, for example, Capmul® MCM (mono-diglyceride of medium chain fatty acids (mainly caprylic and capric)), polyoxylglycerides, isopropyl myristate, isopropyl palmitate, triglycerides, propylene glycol derivatives, glyceryl monooleate, Propylene glycol caprylate, and combinations thereof.

Suitable surfactants or emulsifying agents used in the oil/surfactant/cosurfactant mixture formulation of the present invention include polysorbates (Tween), polysorbate 20, polysorbate 40, polysorbate 60, polysorbate 80, polyoxyethylene castor oil derivatives, caprylocaproyl macrogol glycerides, sorbitan derivatives, polyoxyethylene alkyl ethers, polyoxyethylene sorbitan fatty acids, polyoxyethylene-polyoxypropylene copolymers and other suitable surfactants, for example, long alkyl chain sulfonates/sulfates such as sodium dodecylbenzene sulfonate, sodium lauryl sulfate, and dialkyl sodium sulfosuccinate, quaternary ammonium salts, fatty alcohols such as lauryl, cetyl, and steryl, glycerylesters, fatty acid esters, polyoxyethylene derivatives thereof, and combinations thereof.

Cosurfactants suitable for use with the self-emulsifying excipient formulation of the present invention may contain one or more selected from the group consisting of Transcutol® (Diethylene glycol monoethyl ether, DEGEE), tetraglycol, propylene glycol, diethylene glycol monoethyl ether, and polyethylene glycol and alcohols of intermediate chain length such as hexanol, pentanol, and octanol which are known to reduce the oil/water interface and allow the spontaneous formulation of the emulsion, and combinations thereof.

The method of making a drug delivery system for increasing the bioavailability of a drug and/or pharmaceutical ingredient or formulation by emulsifying the drug with the oil/surfactant/cosurfactant mixture of the present invention includes the steps of solubilizing ticagrelor and additional ingredients in a mixture of surfactant, cosurfactant and oil.

The relative proportions of surfactant and cosurfactant in the oil/surfactant/cosurfactant mixture of the present invention can influence the solubilizing and dissolution properties of the formulation. In some aspects, the emulsion composition may contain about 10% to 50% by weight of oil, 5% to 80% by weight of surfactant and 5% to 80% by weight of the cosurfactant based on the total weight of the emulsion composition and the self-emulsifying drug delivery system characterized in that it contains an emulsion composition and ticagrelor in a weight ratio of 15:1 to 3:1 (w/w) oil/surfactant/cosurfactant mixture to ticagrelor respectively.

A pharmaceutical composition may include a second agent dissolved in an aqueous solution.

In some embodiments, aqueous solutions include, but are not limited to solutions contain water, tris buffer, phosphate buffer, HEPES (4-(2-hydroxyethyl)-1-piperazineethanesulfonic acid) buffer, acetate buffer, citrate buffer, glutamate buffer, ammonium hydroxide with ammonium chloride, carbonic acid with biocarbonate ions, phosphate-buffered saline (PBS), acetic acid, citric acid, oxalic acid, sucrose solutions, or a combination thereof.

In some embodiments, the second agent includes a zinc salt, magnesium salt, a lysine, lecithin, lysine and magnesium salt, lysine and zinc salt or an arginine-containing compound.

In some aspects the LNE formulation of the present invention includes a water insoluble or partially-soluble component pre-dissolved in oil/surfactant/cosurfactant mixture and emulsified in an aqueous solution in a ratio from 1:0.5 to 1:100 (v/v) respectively. In some aspects the LNE formulation of the present invention includes oil/surfactant/cosurfactant mixture emulsified in an aqueous solution in a ratio from 1:0.5 to 1:50 (v/v) respectively. In some aspects the LNE formulation of the present invention includes oil/surfactant/cosurfactant mixture emulsified in an aqueous solution in a ratio from 1:0.5 to 1:20 (v/v) respectively. In some aspects the LNE formulation of the present invention includes oil/surfactant/cosurfactant mixture emulsified in an aqueous solution in a ratio from 1:0.5 to 1:10 (v/v) respectively. In some aspects the LNE formulation of the present invention includes oil/surfactant/cosurfactant mixture emulsified in an aqueous solution in a ratio from 1:0.5 to 1:5 (v/v) respectively. In some aspects the LNE formulation of the present invention includes oil/surfactant/cosurfactant mixture emulsified in an aqueous solution in a ratio from 1:0.5 to 1:2 (v/v) respectively. In some aspects the LNE formulation of the present invention includes oil/surfactant/cosurfactant mixture emulsified in an aqueous solution in a ratio from 1:0.5 (v/v) to 1:1 respectively.

In some aspects the LNE formulation should maintain stability at room temperature for at least 1 month.

In some aspect ticagrelor should remain soluble in the LNE formulation at room temperature for at least 1 month.

### Target Dosing and Population

In some aspects, the present disclosure includes a use or method of treating a subject having an Autism Spectrum Disorders, an intellectual disability, epilepsy, an anxiety disorder, a mood disorder, a disorder of social interaction, irritability, aggression, self-injurious behavior, hyperactivity, inattention, or Fragile X syndrome by administering ticagrelor, an enantiomer thereof, or a pharmaceutically acceptable salt thereof according to the present disclosure in combination with a second agent of the present disclosure. The ticagrelor, an enantiomer thereof, or a pharmaceutically acceptable salt thereof will be co-administered with the second agent. The co-administration may include any suitable manner of administrating the ticagrelor, an enantiomer thereof, or a pharmaceutically acceptable salt thereof with the second agent.

In some aspects, the present disclosure includes a use or method of treating a subject having an Autism Spectrum Disorders, an intellectual disability, epilepsy, an anxiety disorder, a mood disorder, a disorder of social interaction, irritability, aggression, self-injurious behavior, hyperactivity, inattention, or Fragile X syndrome by administering an LNE formulation of the present disclosure. In one aspect, the amount by weight of ticagrelor to be administered per day according to the methods of the present disclosure are less than for conventional ticagrelor regimens, e.g., less than 180 mg/day, less than 120 mg/day, less than 100 mg/day, less than 90 mg/day, less than 80 mg/day, less than 60 mg/day, less than 40 mg/day, less than 30 mg/day, less than 20 mg/day, or less than 10 mg/day

In some aspects, the present disclosure includes a use or method of treating a subject having an neurogenetic, neurodegenerative, neurodevelopmental or neuroinflammatory disorders by administering an LNE formulation of the present disclosure. In one aspect, the amount by weight of ticagrelor to be administered per day according to the methods of the present disclosure are less than for conventional ticagrelor regimens, e.g., less than 180 mg/day, less than 120 mg/day, less than 100 mg/day, less than 90 mg/day, less than 80 mg/day, less than 60 mg/day, less than 40 mg/day, less than 30 mg/day, less than 20 mg/day, or less than 10 mg/day

In some aspects, the methods of the present disclosure will decrease behavioral problems on the Aberrant Behavior Checklist (ABC-C). The ABC-C is a global behavior checklist that measures drug and other treatment effects in people with developmental disabilities. It is made up of five subscales, including Irritability, Lethargy, Inappropriate Speech, Hyperactivity, and Stereotypy based on 58 items that describe various behavioral problems.

In some aspects, the methods of the present disclosure will behavioral problems on the Clinical Global Impression Scale (CGI). The CGI is used by the study psychiatrist to judge the overall clinical condition relative to baseline using the same scale as the CGI-S. The study psychiatrist will rate the improvement from baseline. The CGI consists of a 7-point subjective scale assessing symptom. On this scale, scores of 1, 2, and 3 represent normal, some presence of symptoms, and mild behavior, respectively. A score of 4 represents moderate behavior. Scores of 5, 6, and 7 represent marked, severe, and among the most severe behavior, respectively.

In some aspects, the methods of the present disclosure will behavioral problems on the Modified Overt Aggression Scale (IBR-MOAS). The IBR-MOAS is a questionnaire that includes 5 types of aggression (verbal aggression towards self and others, physical aggression towards objects, self, and others) with four levels of severity for each type of aggression. Only the section assessing the 5 types of aggression will be used for repeat evaluations: Verbal aggression toward others, Verbal aggression toward self, Physical aggression against other people, Physical aggression against objects, Physical aggression against self. The frequency of occurrence of each item are as follows: 0=Never (never happens); 1=Rarely (averages about once a year to once a month); 2=Sometimes (averages about several times a month to several times a week); 3=Often (averages about daily to several times a day); and U (Used to happen but not this past year).

In some aspects, the methods of the present disclosure will behavioral problems on Questions About Behavior Function (QABF) measures. The QABF is an indirect assessment of behavioral function for individuals with developmental disabilities. It contains 25 items. The QABF yields five behavioral function categories: Access to Attention, Escape from Demands, Physical, Access to Tangible, and Nonsocial (i.e., sensory or automatically-maintained). Each question is scored with frequency descriptors of Never, Rarely, Some, and Often. A function is endorsed if the score for a particular function is at or above 4 points or higher.

In certain aspects, the specific dosing regimen involves administering the combination to achieve or maintain a ticagrelor plasma level in a range of about 50-1000 ng/mL, 100-800 ng/mL, about 120-700 ng/mL, about 140-600 ng/mL, about 160-520 ng/mL, about 180-500 ng/mL, about 200-480 ng/mL, about 220-460 ng/mL, about 240-420 ng/mL, about 260-400 ng/mL, or about 500 ng/mL, about 530 ng/mL, about 560 ng/mL, about 580 ng/mL, about 600 ng/mL, or about 620 ng/mL. In some aspects, the dose level is aimed at reaching plasma levels of 1-1000 ng/ml, 2 hours after administration. In some aspects, the ticagrelor or pharmaceutically acceptable salt thereof such that a ticagrelor plasma level of about 50-300 ng/mL is maintained for at least 1, 2, 3, 4, 5, or 6 hours. In some aspects, the ticagrelor or pharmaceutically acceptable salt thereof such that a ticagrelor plasma level of about 30-50 ng/mL is maintained for at least 1, 2, 3, 4, 5, or 6 hours.

Oral dosing of ticagrelor in humans over 18 years is 5, 10, 20, 30, 40, 50, 60, 70, 80, 90, 100, 110, 120, 130, 140, 150, 160, 170, 180, 190, or 200 mg, e.g., 60 to 180 mg ticagrelor, e.g., once or twice daily. In some aspects, the dosage regimen may be increased at weekly intervals according to response

For humans aged 18 and under, doses may be in a range of 0.1-3 mg/kg bodyweight one, two, three, or four times daily.

A method of safely delivering the combination of the present disclosure to a subject can include measuring electrocardiogram, pulse, and blood pressure of the subject before, during, and/or after the administering. Additionally, a method of safely delivering the combination of the present disclosure to a subject can include measuring liver enzyme levels while continuing administration of the combination. In certain aspects, steps can be taken to protect kidney, liver, intestines, or heart if the liver enzyme levels are abnormal, e.g., due to transient transaminase elevations. In some instances, the method further includes the preliminary step of measuring the subject's liver enzyme levels prior to administration. This can be done to establish a baseline liver enzyme level. The method can also include measuring liver enzyme levels at specified time periods after starting the administering step. In some embodiments, the subject's liver enzyme levels can be monitored at 4-hour intervals, 6-hour intervals, 8-hour intervals 12-hour intervals, 24-hour intervals, etc. Alternatively, levels can be checked at about 12 hours, about 24 hours, about 36 hours, about 48 hours, about 60 hours, about 72 hours, about 84 hours, about 96 hours, about 108 hours, about 120 hours, about 132 hours, about 154 hours, about 168 hours, the day the subject is discharged, or a combination thereof.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

The present disclosure includes the following non-limiting aspects:
1) A method of treating a subject diagnosed with an autism spectrum disorder, an intellectual disability, epilepsy, an anxiety disorder, a mood disorder, a disorder of social interaction, irritability, aggression, self-injurious behavior, hyperactivity, inattention, Fragile X syndrome, or a subject diagnosed with elevated TNFα, or with elevated inflammatory cytokine markers of neuroinflammation, encompassing administering a liquid lipid nanocarrier emulsion (LNE) formulation comprising ticagrelor, an enantiomer thereof, or a pharmaceutically acceptable salt thereof dissolved in a mixture comprising an oil, a surfactant and a cosurfactant (an oil/surfactant/cosurfactant mixture) and mixed with a second agent dissolved in an aqueous solution to form a stable emulsion. In some aspects, the ticagrelor oil in water emulsion is stable for at least one month at 25° C./60% RH.
2) The method of aspect 1, wherein the LNE formulation comprises the oil/surfactant/cosurfactant mixture and the aqueous solution in a ratio of 1:0.1 to 1:100.
3) The method of aspect 1 or aspect 2, comprising administering the LNE formulation orally or parenterally.
4) The method of any preceding aspect, comprising administering 10 to 60 mg/day of the ticagrelor, enantiomer or pharmaceutically acceptable salt thereof.
5) The method of any preceding aspect, comprising administering the LNE formulation in an amount sufficient to achieve a ticagrelor plasma level of about 50 to 1000 ng/ml.
6) The method of any preceding aspect, comprising administering the LNE formulation thereof such that said ticagrelor plasma level is maintained for at least 5 hours.
7) The method of any preceding aspect, comprising administering the LNE formulation orally.
8) The method of any preceding aspect, wherein the subject is diagnosed with an anxiety disorder.
9) The method of any preceding aspect, wherein the subject is diagnosed with irritability, aggression, self-injurious behavior, hyperactivity, or inattention.
10) The method of any preceding aspect, wherein the subject is diagnosed with Fragile X syndrome.
11) The method of any preceding aspect, wherein the subject is diagnosed with elevated TNFα.
12) The method of any preceding aspect, wherein the subject is diagnosed with an elevated inflammatory cytokine marker of neuroinflammation.
13) A method of treating a subject diagnosed with an autism spectrum disorder, an intellectual disability, epilepsy, an anxiety disorder, a mood disorder, a disorder of social interaction, irritability, aggression, self-injurious behavior, hyperactivity, inattention, Fragile X syndrome, or diagnosed with elevated TNFα, or diagnosed with elevated inflammatory cytokine marker of neuroinflammation comprising administering a combination of ticagrelor, an enantiomer thereof, or a pharmaceutically acceptable salt thereof dissolved in a mixture comprising an oil, a surfactant and a cosurfactant (an oil/surfactant/cosurfactant mixture) with a second agent dissolved in an aqueous solution wherein:
   the combination comprises the oil/surfactant/cosurfactant mixture in a 1:0.1 to 1:100 by volume to the aqueous solution; and
   the second agent is selected from the group consisting of a magnesium ion containing-compound, a zinc ion containing-compound, L-lysine or a salt thereof, L-arginine or a salt thereof, lecithin, or a combination thereof.
14) The method of any preceding aspect, wherein the second agent is a magnesium ion containing-compound.
15) The method of any preceding aspect, wherein the second agent is a zinc ion containing-compound.
16) The method of any preceding aspect, wherein the second agent is L-lysine or a salt thereof.
17) The method of any preceding aspect, wherein the second agent is L-arginine or a salt thereof.
18) The method of any preceding aspect, wherein the second agent is lecithin.
19) A method of treating a subject diagnosed with an intellectual disability, irritability, aggression, self-injurious behavior, hyperactivity, inattention, Fragile X syndrome, or diagnosed with elevated TNFα, or diagnosed with elevated inflammatory cytokine marker of neuroinflammation comprising administering to the subject a) ticagrelor, an enantiomer thereof, or a pharmaceutically acceptable salt thereof, or ticagrelor, an enantiomer thereof, or a pharmaceutically acceptable salt thereof dissolved in an oil/surfactant/cosurfactant mixture, in combination with b) a second agent dissolved in an aqueous solution wherein:
   a) the oil/surfactant/cosurfactant mixture to aqueous solution ratio is 1:0.1 to 1:100; and
   b) the second agent is selected from the group consisting of a magnesium ion containing-compound, a zinc ion containing-compound, L-lysine or a salt thereof, L-arginine or a salt thereof, lecithin, or a combination thereof.
20) The method of any preceding aspect, wherein the ticagrelor oil in water emulsion are stable for at least one month at 25° C./60% RH.
21) The method of any preceding aspect, comprising, before said administering step, providing:
   c) a first container containing an oil/surfactant/cosurfactant mixture comprising ticagrelor, an enantiomer thereof, or a pharmaceutically acceptable salt thereof dissolved in a mixture comprising an oil, a surfactant and a cosurfactant; and
   d) a second container containing an aqueous solution comprising a magnesium ion containing-compound, a zinc ion containing-compound, L-lysine or a salt thereof, L-arginine or a salt thereof, lecithin, or a combination thereof.
22) The method of any preceding aspect, comprising, before said administering step, providing a dual chamber cartridge, wherein a first chamber of the dual chamber cartridge contains the oil/surfactant/cosurfactant mixture comprising ticagrelor, an enantiomer thereof, or a pharmaceutically acceptable salt thereof dissolved in a mixture comprising an oil, a surfactant and a cosurfactant, and a second chamber of the dual chamber cartridge contains the aqueous solution comprising a magnesium ion containing-compound, a zinc ion containing-compound, L-lysine or a salt thereof, L-arginine or a salt thereof, lecithin, or a combination thereof, wherein the dual chamber cartridge is configured for mixing contents of the first chamber and the second chamber to form the LNE before the administering step.
23) A liquid lipid nanocarrier emulsion (LNE) formulation comprising ticagrelor, an enantiomer thereof, or a pharmaceutically acceptable salt thereof dissolved in a mixture comprising an oil, a surfactant and a cosurfactant (an oil/surfactant/cosurfactant mixture) and a second agent dissolved in an aqueous solution, wherein the second agent is selected from the group consisting of a magnesium ion containing-compound, a zinc ion containing-compound, L-lysine or a salt thereof, L-arginine or a salt thereof, lecithin, or a combination thereof.
24) The LNE formulation of aspect 23, comprising the oil/surfactant/cosurfactant mixture in a 1:0.1 to 1:100 ratio by volume to the aqueous solution.
25) The LNE formulation of aspect 23 or 24, wherein the oil is selected from a caprylic acid glyceride, a capric acid glyceride, a polyoxylglyceride, an isopropyl myristate, an isopropyl palmitate, a triglyceride, a propylene glycol oil, or glyceryl monooleate.
26) The LNE formulation of any preceding aspect, wherein the surfactant is selected from a polysorbate, a polyoxyethylene castor oil derivatives, a caprylocaproyl macrogol glyceride, a sorbitan, a polyoxyethylene alkyl ether, a polyoxyethylene sorbitan fatty acid, a polyoxyethylene-polyoxypropylene copolymer, an alkyl sulfonate, an alkyl sulfate a quaternary ammonium salt, a fatty alcohol, a glycerylester, a fatty acid ester, or a polyoxyethylene.
27) The LNE formulation of any preceding aspect, wherein the cosurfactant is selected from a tetraglycol, a propylene glycol, a diethylene glycol monoethyl ether, a polyethylene glycol, hexanol, pentanol, or octanol.
28) The LNE formulation of any preceding aspect, wherein the LNE formulation comprises 5% to 50% by weight of oil, 5% to 80% by weight of surfactant and 5% to 80% by weight of the cosurfactant based on the total weight of the oil/surfactant/cosurfactant mixture.
29) The LNE formulation of any preceding aspect, wherein the LNE formulation comprises 10% to 20% by weight of oil, 20% to 60% by weight of surfactant and 15% to 60% by weight of the cosurfactant based on the total weight of the oil/surfactant/cosurfactant mixture.
30) The LNE formulation of any preceding aspect, wherein the LNE formulation comprises a weight ratio of 15:1 to 3:1 (w/w) oil/surfactant/cosurfactant mixture to ticagrelor.
31) The LNE formulation of any preceding aspect, wherein the LNE formulation comprises a weight ratio of 10:1 to 5:1 (w/w) oil/surfactant/cosurfactant mixture to ticagrelor.
32) The LNE formulation of any preceding aspect, comprising 10-90 mg of the ticagrelor, enantiomer thereof, or pharmaceutically acceptable salt thereof.
33) The LNE formulation of any preceding aspect, comprising 15-80 mg of the ticagrelor, enantiomer thereof, or pharmaceutically acceptable salt thereof.
34) The LNE formulation of any preceding aspect, comprising 20-70 mg of the ticagrelor, enantiomer thereof, or pharmaceutically acceptable salt thereof.
35) The LNE formulation of any preceding aspect, comprising 25-50 mg of the ticagrelor, enantiomer thereof, or pharmaceutically acceptable salt thereof.
36) The LNE formulation of any preceding aspect, wherein the weight ratio of the oil:surfactant ranges from 1:0.5 to 1:80.
37) The LNE formulation of any preceding aspect, wherein the weight ratio of the oil:cosurfactant ranges from 1:0.4 to 1:50.
38) The LNE formulation of any preceding aspect, wherein the weight percentage of the ticagrelor, enantiomer thereof, or pharmaceutically acceptable salt thereof to the oil/surfactant/cosurfactant mixture ranges from 1:3 to 1:15 respectively.
39) The LNE formulation of any preceding aspect, wherein the weight percentage of the ticagrelor, enantiomer thereof, or pharmaceutically acceptable salt thereof to the oil/surfactant/cosurfactant mixture ranges from 1:5 to 1:10 respectively.
40) The LNE formulation of any preceding aspect, wherein the LNE formulation comprises the second agent in a concentration of 1 to 300 mg/ml.
41) The LNE formulation of any preceding aspect, wherein the LNE formulation comprises the second agent in a concentration of 5 to 200 mg/ml.
42) The LNE formulation of any preceding aspect, wherein the LNE formulation comprises the second agent in a concentration of 10 to 100 mg/ml.
43) The LNE formulation of any preceding aspect, wherein the LNE formulation comprises the second agent in a concentration of 15 to 80 mg/ml.
44) The LNE formulation of any preceding aspect, wherein the LNE formulation comprises the second agent in a concentration of 20 to 60 mg/ml.
45) The LNE formulation of any preceding aspect, wherein the LNE formulation comprises the second agent in a concentration of 25 to 50 mg/ml.
46) The LNE formulation of any preceding aspect, wherein the LNE formulation comprises the second agent in a concentration of 30 to 40 mg/ml.
47) The LNE formulation of any preceding aspect, wherein the molar ratio of the ticagrelor, enantiomer thereof, or pharmaceutically acceptable salt thereof to the second agent in the LNE formulation ranges from 1:0.1 to 1:100 (mol/mol).
48) The LNE formulation of any preceding aspect, wherein the molar ratio of the ticagrelor, enantiomer thereof, or pharmaceutically acceptable salt thereof to the second agent in the LNE formulation ranges from 1:1 to 1:60 (mol/mol).
49) The LNE formulation of any preceding aspect, wherein the molar ratio of the ticagrelor, enantiomer thereof, or pharmaceutically acceptable salt thereof to the second agent in the LNE formulation ranges from 1:5 to 1:40 (mol/mol).
50) The LNE formulation of any preceding aspect, wherein the molar ratio of the ticagrelor, enantiomer thereof, or pharmaceutically acceptable salt thereof to the second agent in the LNE formulation ranges from 1:10 to 1:30 (mol/mol).
51) The LNE formulation of any preceding aspect, wherein the molar ratio of the ticagrelor, enantiomer thereof, or pharmaceutically acceptable salt thereof to the second agent in the LNE formulation ranges from 1:15 to 1:20 (mol/mol).
52) A kit comprising:
   a) a first container containing an oil/surfactant/cosurfactant mixture comprising ticagrelor, an enantiomer thereof, or a pharmaceutically acceptable salt thereof dissolved in a mixture comprising an oil, a surfactant and a cosurfactant; and
   b) a second container containing an aqueous solution comprising a magnesium ion containing-compound, a zinc ion containing-compound, L-lysine or a salt thereof, L-arginine or a salt thereof, lecithin, or a combination thereof.
53) A dual chamber cartridge, wherein a first chamber of the dual chamber cartridge contains an oil/surfactant/cosurfactant mixture comprising ticagrelor, an enantiomer thereof, or a pharmaceutically acceptable salt thereof dissolved in a mixture comprising an oil, a surfactant and a cosurfactant, and a second chamber of the dual chamber cartridge contains an aqueous solution comprising a magnesium ion containing-compound, a zinc ion containing-compound, L-lysine or a salt thereof, L-arginine or a salt thereof, lecithin, or a combination thereof, wherein the dual chamber cartridge is configured for mixing contents of the first chamber and the second chamber to form a LNE formulation.

Other aspects, advantages, and modifications are within the scope of claims below.

### EXAMPLES

The following examples are intended to exemplify the present disclosures and are not limitations of the claimed invention. All molecules, compositions, methods, assays, and results disclosed in the examples form non-limiting parts of the present disclosure.

### Example 1: Ticagrelor/Zinc Combination Ameliorates Autism-Like Symptoms and Neuroinflammation in the Valproic Acid (VPA) Model of Autism

Exposure to valproic acid (VPA) during pregnancy has been demonstrated to increase the risk of autism in children. Furthermore, rodents prenatally exposed to this drug display behavioral phenotypes characteristics of the human autism condition. This model might better represent the many cases of idiopathic autism which are of environmental/epigenetic origins than do transgenic models carrying mutations in single autism-associated genes. The VPA model is an environmentally triggered model with strong construct and clinical validity. The VPA-induced rodent model is an art-recognized and well-established and widely utilized animal model to test putative efficacy of pharmacological agents on behavioral and social elements in the autistic spectrum for humans.

C57BL/6J mice were obtained at 7-8 weeks of age and were mated, with pregnancy confirmed by the presence of a vaginal plug on embryonic day 0 (E0). On E12.5, pregnant females received a single intraperitoneal injection of sodium salt of valproic acid (VPA, 500 mg/kg) dissolved in saline (SAL). Control females received an equal volume of SAL only. Day of birth was recorded as PO (postnatal day zero). The offspring were weaned on P21 (postnatal day 21) and the male offspring were housed in groups of 4-5. Experiments were carried out on male offspring. The mice were housed in a temperature- and humidity-controlled environment with ad libitum access to food and water. Animals were maintained on a 12 hr light/dark schedule, with lights on at 8 A.M.

The male offspring of VPA-treated mice were divided into 4 groups (n=8-9 per group): VPA-Vehicle)+50 μg of ZnSO4, VPA-ticagrelor (0.3 mg/kg)+50 μg of ZnSO4, VPA-ticagrelor (10 mg/kg)+50 μg of ZnSO4 and VPA-ticagrelor (30 mg/kg)+50 μg of ZnSO4. The male offspring of SAL-treated mice were divided into 2 groups (n=10 per group): SAL-Vehicle and SAL-ticagrelor (30 mg/kg)+50 μg of ZnSO4. From the sixth week to eight week of age, the mice received three once-weekly injections of either saline (5 μl/g ip) or ticagrelor (i.p.). Twenty-four hours after the last injection, the three chamber test was performed. The animals were then sacrificed and proinflammatory cytokines, IL-6 and TNF-α, were evaluated in whole brain and serum. Blood samples were collected via retro-orbital puncture, and centrifuged at 2000 g for 20 min, followed by supernatant (serum) isolation. The brain was removed after PBS perfusion via heart under anesthesia. The whole brain was then dissected and homogenized in PBS buffer with protease and phosphatase inhibitors and centrifuged (10,000 rpm, 5 min). The supernatant was collected and assayed for IL-6 and TNF-α levels according to ELISA Kit protocols (Proteintech, Cat No. KE10007 and KE10002 respectively).

### Behavioral Assay-Three Chamber Test

The three-chamber test assesses cognition in the form of general sociability and interest in social novelty in a rodent model of CNS disorders. This test measures the sociability, the propensity to spend time with another mouse, as compared to time spent alone in an identical but empty chamber.

The apparatus was an acrylic box (length: 62 cm, width: 41 cm, height: 30.5 cm) divided into three chambers. A 5 cm×5 cm opening was made on the partitions so that mice could freely explore the three chambers. Mouse was first placed into the center chamber of the apparatus and allowed 5 min for habituation. Following the completion of the acclimatization period the test mouse was removed to a holding cage, and the bedding material was redistributed. Then a novel mouse enclosed in a tiny cage was placed in one lateral chamber (social area) and an empty tiny cage was placed in the other lateral chamber as a novel object (non-social area). Mouse was first placed into the center chamber and allowed to freely explore the chambers for 10 min and the time spent in each of the three areas (social, center, and non-social areas) was recorded.

Statistical Analyses: Statistical analyses for the data were performed using one-way or two-way RM ANOVA (SigmaStat 3.1). Post-hoc comparisons were performed with the Tukey HSD method. If data were not normally distributed, Kruskal-Wallis one way ANOVA on ranks followed by the Dunn's method was used. All data were represented as mean±SEM. Significant level was set at p<0.05.

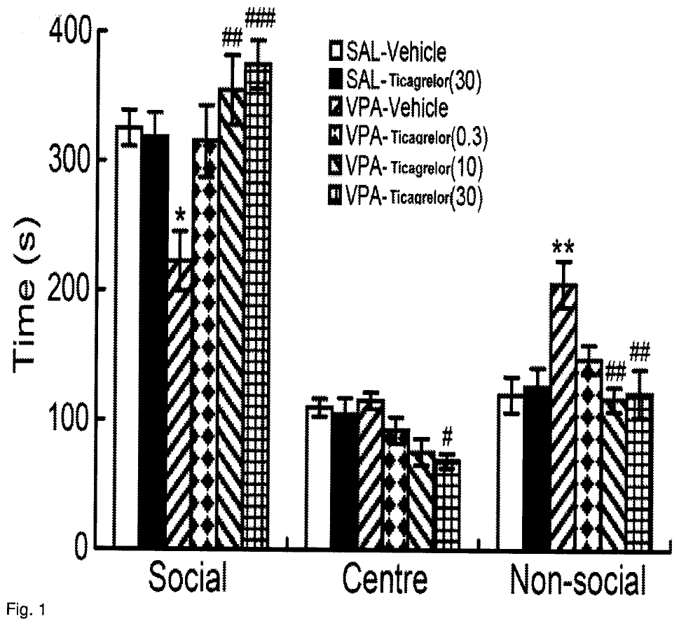

Results: Effects on behavior: A one-way ANOVA of the time spent in social area revealed a significant difference among the six groups (F (5, 48)=5.201, p<0.001). Post hoc comparisons showed that compared with the SAL-Vehicle group, the VPA-Vehicle group showed a significant decrease of the time spent in social area (p<0.05) (FIG. 1, left panel). Compared with the VPA-Vehicle group, both the VPA-ticagrelor (10 mg/kg) and VPA-ticagrelor (30) groups showed a significant increase of the time spent in social area (p<0.01 and p<0.001). There was no significant difference in the time spent in social area between the SAL-Vehicle group and the SAL-ticagrelor (30) group (p>0.05).

A one-way ANOVA of the time spent in center area revealed a significant difference among the six groups (F(5, 48)=3.821, p<0.01). Post hoc comparisons showed that compared with the VPA-Vehicle group, the VPA-ticagrelor (30) group showed a significant decrease of the time spent in center area (p<0.05) (FIG. 1, center panel).

A one-way ANOVA of the time spent in non-social area revealed a significant difference among the six groups (F(5, 48)=5.230, p<0.001). Post hoc comparisons showed that compared with the SAL-Vehicle group, the VPA-Vehicle group showed a significant increase of the time spent in non-social area (p<0.01) (FIG. 1, right panel). Compared with the VPA-Vehicle group, both the VPA-ticagrelor (10 mg/kg) and VPA-ticagrelor (30 mg/kg) groups showed a significant decrease of the time spent in non-social area (both, p<0.01). There was no significant difference in the time spent in non-social area between the SAL-Vehicle group and the SAL-ticagrelor (30 mg/kg) group (p>0.05).

Results: Effects on Biochemical Neuroinflammatory Markers:

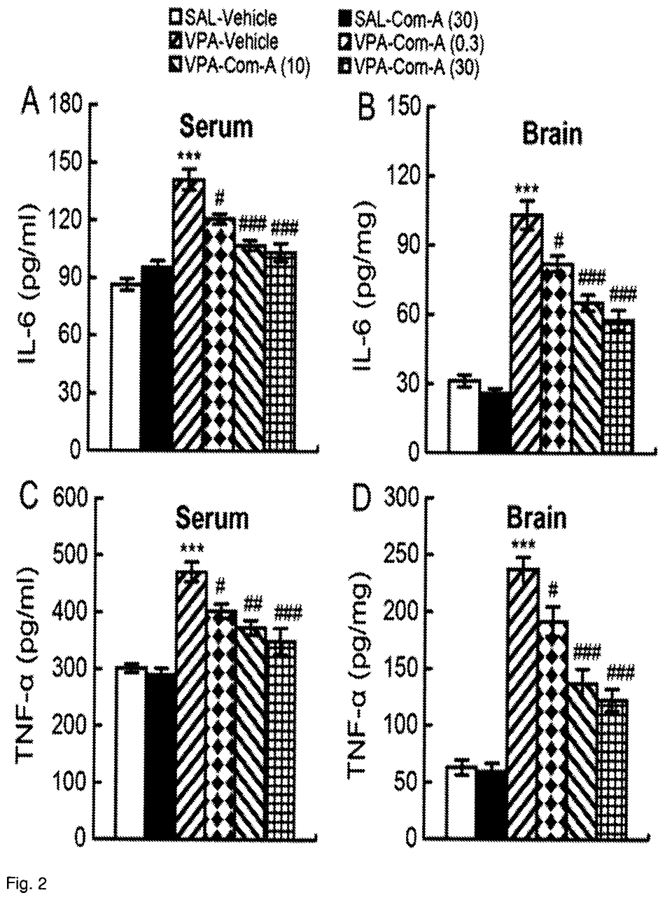

FIG. 2 shows the effects of Ticagrelor/zinc (Com-A) on neuroinflammation in a mouse model of autism.

A one-way ANOVA analysis of the level of IL-6 in serum revealed a significant difference among the six groups (F (5, 20)=24.461, p<0.001) (FIG. 2, panel A). Post hoc comparisons showed that compared with the SAL group, the VPA group showed a significant increase of the level of IL-6 in serum (p<0.001). Compared with the VPA group, all VPA-Ticagrelor groups (0.3, 10, and 30 mg/kg) showed a significant decrease of the level of IL-6 in serum (p<0.05, p<0.001 and p<0.001 respectively). There was no significant difference in the level of IL-6 in serum between the SAL and the SAL-Ticagrelor (30 mg/kg) groups (p>0.05).

A one-way ANOVA analysis of the level of IL-6 in the brain revealed a significant difference among the six groups (F (5, 20)=54.227, p<0.001) (FIG. 2, panel B). Post hoc comparisons showed that compared with the SAL group, the VPA group showed a significant increase of the level of IL-6 in the brain (p<0.001). Compared with the VPA group, VPA-Ticagrelor groups (Ticagrelor 0.3, 10, and 30 mg/kg) showed a significant decrease of the level of IL-6 in the brain (p<0.05, p<0.001 and p<0.001, respectively). There was no significant difference in the level of IL-6 in the brain between the SAL group and the SAL-Ticagrelor (30 mg/kg) group (p>0.05).

A one-way ANOVA analysis of the level of TNF-α in serum revealed a significant difference among the six groups (F (5, 20)=22.025, p<0.001) (FIG. 2, panel C). Post hoc comparisons showed a significant increase of the level of TNF-α in serum (p<0.001) in the VAP group compared with the SAL group. Compared with the VPA group, all VPA-Ticagrelor groups (Ticagrelor 0.3, 10, and 30 mg/kg) showed a significant decrease of the level of TNF-α in serum (p<0.05, p<0.01 and p<0.001 respectively). There was no significant difference in the level of TNF-α in serum between the SAL group and the SAL-Ticagrelor (30 mg/kg) group (p>0.05).

A one-way ANOVA analysis of the level of TNF-α in the brain revealed a significant difference among the six groups (F (5, 20)=52.110, p<0.001) (FIG. 2, panel D). Post hoc comparisons showed that compared with the SAL-group, the VPA-group showed a significant increase of the level of TNF-α in the brain (p<0.001). Compared with the VPA group, the VPA-VPA-Ticagrelor groups (0.3, 10, and 30 mg/kg) showed a significant decrease of the level of TNF-α in the brain (p<0.05, p<0.01 and p<0.001). There was no significant difference in the level of TNF-α in the brain between the SAL group and the SAL-Ticagrelor (30 mg/kg) group (p>0.05).

Conclusions: Three once-weekly administration of ticagrelor and zinc salt attenuated the deficit in sociability, an autism-relevant symptom, displayed by the in utero exposed to VPA C57BL/6J mice. Impairments were observed in the 3-chamber test of the VPA group compared to the control mice. In contrast, ticagrelor-treated groups, at doses of 10 and 30 mg/kg, presented a significant increase in time spent near the cage with the stimulus mouse (“social” area). Additionally, ticagrelor/zinc administration resulted in a significant reduction in neuroinflammatory markers, an autism-associated pathology, in all doses tested. The data show a postnatal therapeutic effect of ticagrelor/zinc in an ASD animal model.

### Comparative Example 2: Zinc Salts are Insoluble in an Oil/Surfactant/Cosurfactant Mixture

Studies were performed to introduce a 100 mg/ml of zinc salt in a 100 mg/ml ticagrelor in an oil/surfactant/cosurfactant mixture. Ticagrelor was dissolved (102 mg/ml) in oil/surfactant/cosurfactant mixture made of Campul® MCM (oil) (mono-diglyceride of medium chain fatty acids (mainly caprylic and capric)), Montanox™ 80PHA(surfactant) (polysorbate 80, hydrophilic non-ionic surfactant) and Transcutol® (cosurfactant) (Diethylene glycol monoethyl ether, DEGEE) in a 4:21.5:14.5 w/w ratio.

Observations

Ticagrelor in this formulation was visually soluble at 102 mg/ml. Zinc citrate was then added to the ticagrelor-oil/surfactant/cosurfactant mixture preparation at 1-100 mg/ml concentration and was insoluble at any of the concentrations tested.

Formulation of oil/surfactant/cosurfactant mixture with zinc citrate was stopped because the solubility was unsatisfactory.

Other zinc salts, zinc caprylate zinc stearate and zinc gluconate did not dissolve in oil/surfactant/cosurfactant mixture as well even at low concentrations and with longer steering time (up to 24 h). Changing the cosurfactant to Lauroglycol™ (propylene glycol monolaurate) or the surfactant to Kolliphor® (a non-ionic solubilizer and emulsifier made by reacting castor oil with ethylene oxide in a molar ratio of 1:35) did not improve zinc solubility.

### Example 3: LNE Preparation

A oil/surfactant/cosurfactant mixture was prepared by mixing an oil (Capmul® MCM), a surfactant (Tween® 80) (polysorbate 80) and a cosurfactant (Transcutol® HP). The mixture was gently stirred to make a uniform solution before adding ticagrelor (referred to as “Mix A”).

Mix A - Oil/surfactant/cosurfactant solution

| Ingredients | Amount (g) | Amount (w/w %) |
|---|---|---|
| Capmul ® MCM (mg) | 0.5 | 8.9 |
| Tween ® 80 (mg) | 2.7 | 48.2 |
| Transcutol ® HP (mg) | 1.8 | 32.1 |
| Ticagrelor | 0.6 | 10.7 |

Observation: Ticagrelor in this formulation was visually soluble.

Zinc gluconate was mixed in distilled water to obtain a 35 mg/ml solution (referred to as “Mix B”).

Mix B - Zinc gluconate in water solution

| Ingredients | Amount |
|---|---|
| Zinc gluconate (mg) | 350 mg |
| Distilled water | 10 ml |

Observation: Zinc gluconate was visibly soluble at this concentration.

LNE preparation

Mix A and Mix B were combined in a 1:9 ratio (vol/vol) respectively and the mixture was emulsified by Vortex for 30 seconds until visibly clear. The resulting emulsion was filtered through a 0.45 μm polyvinylidene fluoride (PVDF) membrane (Millipore, Bengaluru, Karnataka, India) and the pass-thru liquid LNE was collected. Final Ticagrelor concentration in the LNE as measured by LC-MS was 10 mg/ml. Final zinc gluconate concentration in the LNE (estimated) was 32 mg/ml. The Ticagrelor/zinc ion ratio in the LNE was 2:1 mole/mole.

### Example 4: LNE Preparation 2

A oil/surfactant/cosurfactant mixture is prepared by mixing an oil (Capryol 90®, Propylene Glycol Caprylate), a surfactant (Cremophor EL®, PEG-35 castor oil) (polysorbate 80) and a cosurfactant (Transcutol® HP). The mixture is gently stirred to make a uniform solution before adding ticagrelor (referred to as “Mix A”).

Mix A - Oil/surfactant/cosurfactant solution

| Ingredients | Amount (g) | Amount (w/w %) |
|---|---|---|
| Capryol 90 (mg) | 0.9 | 16.1 |
| Cremophor EL (mg) | 2.3 | 41.1 |
| Transcutol ® HP (mg) | 1.4 | 25.0 |
| Ticagrelor | 1.0 | 17.8 |

Ticagrelor in this formulation is visually soluble.

Zinc gluconate is mixed in distilled water to obtain a 40 mg/ml solution (referred to as “Mix B”).

Mix B - Zinc gluconate in water solution

| Ingredients | Amount |
|---|---|
| Zinc gluconate (mg) | 400 mg |
| Distilled water | 10 ml |

Zinc gluconate is visibly soluble at this concentration.

LNE Preparation

Mix A and Mix B are combined in a 1:9 ratio (vol/vol) respectively and the mixture is emulsified by Vortex for 30 seconds until visibly clear. The resulting emulsion is filtered through a 0.45 μm polyvinylidene fluoride (PVDF) membrane (Millipore, Bengaluru, Karnataka, India) and the pass-thru liquid LNE is collected. Final Ticagrelor concentration in the LNE as measured by LC-MS is 10 mg/ml. Final zinc gluconate concentration in the LNE (estimated) is 36 mg/ml. The Ticagrelor/zinc ion ratio in the LNE is 2.4:1 mole/mole.

### Example 5: LNE Preparation 3

A oil/surfactant/cosurfactant mixture is prepared by mixing an oil (Lauroglycol 90®, Propylene Glycol Laurate), a surfactant (Tween 20, polysorbate 20) and a cosurfactant (Tetraglycol). The mixture is gently stirred to make a uniform solution before adding ticagrelor (referred to as “Mix A”).

Mix A - Oil/surfactant/cosurfactant solution

| Ingredients | Amount (g) | Amount (w/w %) |
|---|---|---|
| Lauroglycol 90 (mg) | 0.3 | 5.35 |
| Tween 20 (mg) | 3.5 | 62.5 |
| Tetraglycol (mg) | 1.5 | 26.8 |
| Ticagrelor | 0.3 | 5.35 |

Ticagrelor in this formulation is visually soluble.

Zinc gluconate is mixed in distilled Phosphate buffer saline (PBS) to obtain a 20 mg/ml solution (referred to as “Mix B”).

Mix B - Zinc gluconate in water solution

| Ingredients | Amount |
|---|---|
| Zinc gluconate (mg) | 200 mg |
| PBS | 10 ml |

Zinc gluconate is visibly soluble at this concentration.

LNE Preparation

Mix A and Mix B are combined in a 1:9 ratio (vol/vol) respectively and the mixture is emulsified by Vortex for 30 seconds until visibly clear. The resulting emulsion is filtered through a 0.45 μm polyvinylidene fluoride (PVDF) membrane (Millipore, Bengaluru, Karnataka, India) and the pass-thru liquid LNE is collected. Final Ticagrelor concentration in the LNE as measured by LC-MS is 10 mg/ml. Final zinc gluconate concentration in the LNE (estimated) is 18 mg/ml. The Ticagrelor/zinc ion ratio in the LNE is 1.4:1 mole/mole.

### Example 6: Zinc Salts do not Affect Ticagrelor Bioavailability

Objective: To compare the effect of zinc on ticagrelor plasma and brain bioavailability following a single oral (PO) administration in male Sprague Dawley rats.

Experimental Procedure:

Group Allocation and Treatment

| | | | Formulation | Time point |
|---|---|---|---|---|
| | | | strength | (h) 5 |
| | Treatment | Dose, ticagrelor, | ticagrelor, zinc | animals per |
| Group | (Dose: mg/kg) | zinc (mg/kg) | (mg/mL) | time point |
| 1 | Ticagrelor + | 25, 25 | 10, 32 | 0.75 h |
| | Zinc citrate | | | N = 2 |
| | | | | 2 h |
| | | | | N = 2 |
| 2 | Ticagrelor | 25 | 10 | 0.75 h |
| | | | | N = 2 |
| | | | | 2 h |
| | | | | N = 2 |

All rats were dosed orally with ticagrelor powder (25 mg/kg body weight) and zinc citrate powder (25 mg/kg body weight) or ticagrelor powder alone (25 mg/kg body weight) in a PBS suspension via oral gavage. Plasma, CSF, and brain samples were collected from each animal using standard procedures at 0.75 h or 2 hours after dosing.

Bioanalysis of ticagrelor was performed in samples from Plasma, CSF and Brain by the fit-for-purpose analytical method using LC-MS/MS.

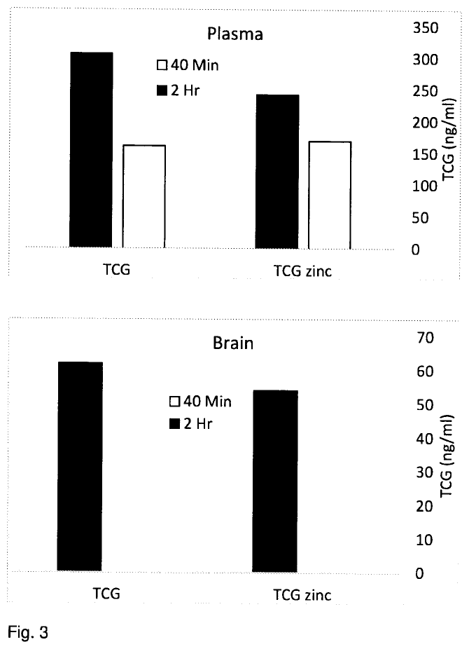

Results and conclusion: FIG. 3 shows the effects of Zinc on ticagrelor (TCG) bioavailability in the plasma and brain of rats. Zinc does not affect ticagrelor bioavailability in plasma or brain of rats (FIG. 3).

### Example 7: Bioavailability of Ticagrelor LNE Formulation Containing Zinc Salt

Objective: To compare the plasma and brain bioavailability of Ticagrelor/zinc in LNE formulation (see Example 3) versus Ticagrelor/zinc suspension in water+0.5% carboxymethylcellulose (CMC) and ticagrelor/zinc suspension in PBS following a single oral (PO) administration in male Sprague Dawley rats.

### Experimental Procedure

### Group Allocation and Treatment

| | | | Formulation | |
|---|---|---|---|---|
| | | Dose, | strength | Time point, |
| | Treatment | ticagrelor, | ticagrelor, zinc | animals per |
| Group | (Dose: mg/kg) | zinc (mg/kg) | (mg/mL) | time point |
| 1 | Ticagrelor + Zinc | 30, 96 | 10, 32 | 0.66 h, N = 5 |
| | gluconate in LNE | | | 2 h, N = 5 |
| | | | | 8 h, N = 5 |
| 2 | Ticagrelor + Zinc | 30, 96 | 10, 32 | 0.66 h, N = 5 |
| | gluconate in 0.5% | | | 2 h, N = 5 |
| | CMC solution | | | 8 h, N = 5 |
| 3 | Ticagrelor + Zinc | 30, 96 | 10, 32 | 2 h, N = 3 |
| | gluconate in PBS | | | |

### Dose Administration

All rats were dosed with LNE containing ticagrelor (30 mg/kg body weight) and zinc citrate (96 mg/kg body weight) or Ticagrelor/zinc suspension in water+0.5% carboxymethylcellulose (CMC) and ticagrelor/zinc suspension in PBS via oral gavage. Plasma, CSF, and brain samples were collected from each animal using standard procedures at 0.66 h, 2 or 8 hours after dosing.

Bioanalysis of ticagrelor was performed in samples from Plasma and Brain by fit-for-purpose analytical method using LC-MS/MS.

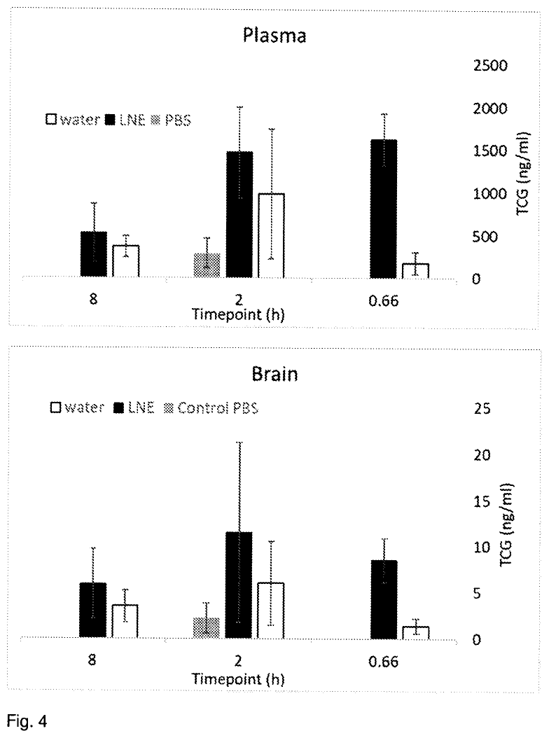

Results: FIG. 4 shows the bioavailability of ticagrelor (TCG) LNE formulation versus water or PBS formulations. The bioavailability of ticagrelor was greatly enhanced in the LNE compared to controls (FIG. 4).

Conclusion: Formulation of Ticagrelor plus zinc in LNE dramatically increases the bioavailability of Ticagrelor in both the brain and plasma of rats.

### Example 8

### LNEs S1-S31 Further Exemplify Aspects of the Present Disclosure:

| | S1 | S2 | S3 | S4 | S5 | S6 | S7 |
|---|---|---|---|---|---|---|---|
| Active material | | | | | | | |
| Ticagrelor free base or in | X | X | X | X | X | X | X |
| complex with (1) | | | | | | | |
| Magnesium salt with (2) | X | | | X | | | |
| Zinc salt with (2) | | X | | | X | X | |
| L-Lysine or L-arginine | | | X | | X | | X |
| lecithin | | | | | | | X |
| Oil Component | | | | | | | |
| Caprylic acid glyceride | X | | | | X | X | |
| and/or Capric acid | | | | | | | |
| glyceride | | | | | | | |
| isopropyl myristate and/or | | X | | | | | X |
| isopropyl palmitate | | | | | | | |
| glyceryl monooleate | | | X | | | | |
| Triglyceride-containing oil | | | | X | | | |
| Surfactant Component | | | | | | | |
| Polysorbate 80 | X | X | X | X | | | |
| polyoxyethylene castor oil | | | | | X | | X |
| caprylocaproyl macrogol | | | | | | X | |
| glycerides | | | | | | | |
| Cosurfactant Component | | | | | | | |
| Diethylene glycol | X | X | X | X | X | X | X |
| monoethyl ether | | | | | | | |
| tetraglycol | | | | | | | |
| propylene glycol | | | | | | | |

| | S8 | S9 | S10 | S11 | S12 | S13 | S14 | S15 |
|---|---|---|---|---|---|---|---|---|
| Active material | | | | | | | | |
| Ticagrelor free base or in | X | X | X | X | X | X | X | X |
| complex with (1) | | | | | | | | |
| Magnesium salt with (2) | X | X | | X | | | X | |
| Zinc salt with (2) | | | X | | X | | | X |
| L-Lysine or L-arginine | X | | | | | | | |
| lecithin | | | | | | X | X | X |
| Oil Component | | | | | | | | |
| Caprylic acid glyceride | | | | | | | X | |
| and/or Capric acid | | | | | | | | |
| glyceride | | | | | | | | |
| isopropyl myristate and/or | | | | | | | | X |
| isopropyl palmitate | | | | | | | | |
| glyceryl monooleate | X | X | X | | | | | |
| Triglyceride-containing oil | | | | X | X | X | | |
| Surfactant Component | | | | | | | | |
| Polysorbate 80 | X | | | X | | | X | X |
| polyoxyethylene castor oil | | X | | | X | | | |
| caprylocaproyl macrogol | | | X | | | X | | |
| glycerides | | | | | | | | |
| Cosurfactant Component | | | | | | | | |
| Diethylene glycol | X | X | X | X | X | X | | |
| monoethyl ether | | | | | | | | |
| tetraglycol | | | | | | | X | |
| propylene glycol | | | | | | | | X |
| | S16 | S17 | S18 | S19 | S20 | S21 | S22 | S23 |
| Active material | | | | | | | | |
| Ticagrelor free base or in | X | X | X | X | X | X | X | X |
| complex with (1) | | | | | | | | |
| Magnesium salt with (2) | X | X | | X | | | X | |
| Zinc salt with (2) | | | X | | X | | | X |
| L-Lysine or L-arginine | X | | | | | | | |
| lecithin | | | | | | X | X | X |
| Oil Component | | | | | | | | |
| Caprylic acid glyceride | | | | | X | X | | |
| and/or Capric acid | | | | | | | | |
| glyceride | | | | | | | | |
| isopropyl myristate and/or | | | | | | | X | X |
| isopropyl palmitate | | | | | | | | |
| glyceryl monooleate | X | X | | | | | | |
| Triglyceride-containing oil | | | X | X | | | | |
| Surfactant Component | | | | | | | | |
| Polysorbate 80 | X | X | X | X | | | | |
| polyoxyethylene castor oil | | | | | X | X | X | X |
| caprylocaproyl macrogol | | | | | | | | |
| glycerides | | | | | | | | |
| Cosurfactant Component | | | | | | | | |
| Diethylene glycol | | | | | | | | |
| monoethyl ether | | | | | | | | |
| tetraglycol | X | | X | | X | | X | |
| propylene glycol | | X | | X | | X | | X |
| | S24 | S25 | S26 | S27 | S28 | S29 | S30 | S31 |
| Active material | | | | | | | | |
| Ticagrelor free base or in | X | X | X | X | X | X | X | X |
| complex with (1) | | | | | | | | |
| Magnesium salt with (2) | X | X | | X | | | X | |
| Zinc salt with (2) | | | X | | X | | | X |
| L-Lysine or L-arginine | X | | | | | | | |
| lecithin | | | | | | X | X | X |
| Oil Component | | | | | | | | |
| Caprylic acid glyceride | | | | | X | X | | |
| and/or Capric acid | | | | | | | | |
| glyceride | | | | | | | | |
| isopropyl myristate and/or | | | | | | | X | X |
| isopropyl palmitate | | | | | | | | |
| glyceryl monooleate | X | X | | | | | | |
| Triglyceride-containing oil | | | X | X | | | | |
| Surfactant Component | | | | | | | | |
| Polysorbate 80 | | | | | | | | |
| polyoxyethylene castor oil | X | X | X | X | | | | |
| caprylocaproyl macrogol | | | | | X | X | X | X |
| glycerides | | | | | | | | |
| Cosurfactant Component | | | | | | | | |
| Diethylene glycol | | | | | | | | |
| monoethyl ether | | | | | | | | |
| tetraglycol | X | | X | | X | | X | |
| propylene glycol | | X | | X | | X | | X |

(1) In complex with: hydrochloric acid, pyrophosphoric acid, tauric acid, sulfuric acid, acetic acid, caprylic acid, carbonic acid, citric acid, ascorbic acid, threonic acid, tartaric acid, a malic acid, fumaric acid, gluconic acid, succinic acid, lactic acid, a glyceric acid, a glyceraldehyde, or an alpha-hydroxy-carboxylic acid.
(2) Salt Form: sulphate, acetate, threonate, tartarate, glycerate, caprylate, citrate, chloride, sulfate, lactate, carbonate, malate, taurate, gluconate, succinate, or pyrophosphate. Acids: tartaric acid, glyceric acid, threonic acid, caprylic or gluconic acid.

### Example 9 LNE Formulation in a Kit

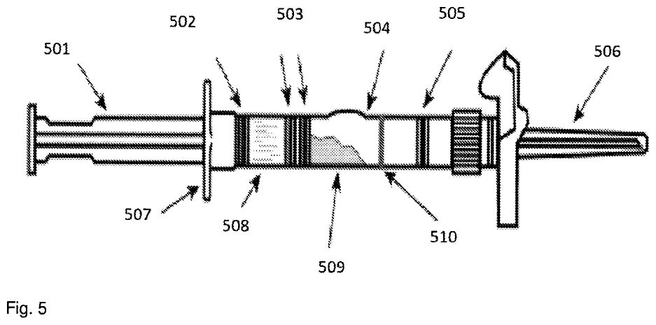

Cartridges Double Chamber (also referred to as dual chamber cartridge device) is a device containing two drug components in two isolated chambers separated by a plunger and a plunger rod. A bypass within the glass allows contents to mix before the drug is delivered. When pressing the plunger rod, the middle plunger moves into a bypass position that allows mixing of compound A and compound B (FIG. 5). The device allows safe and high accuracy mixing by the patient or a caregiver. Once mixed the drugs can be delivered orally or injected. (Rahul G. Ingle, Wei-Jie Fang International Journal of Pharmaceutics Volume 597, 15 Mar. 2021, 120314). FIG. 5 shows the plunger rod (501); end stopper (502); middle stoppers (503); barrel (504); front stopper (505); needle and cap (506), finger grip (507), compound A (508), compound B (509), and blue line (510).

The Cartridges Double Chamber is filled with the ticagrelor in oil/surfactant/cosurfactant at position A and the second agent in aqueous solution in chamber B. Mixing is done immediately before use.

Compound A - Oil/surfactant/cosurfactant solution

| Ingredients | Amount (g) | Amount (w/w %) |
|---|---|---|
| Capmul ® MCM (mg) | 0.5 | 8.9 |
| Tween ® 80 (mg) | 2.7 | 48.2 |
| Transcutol ® HP (mg) | 1.8 | 32.1 |
| Ticagrelor | 0.6 | 10.7 |

Zinc gluconate is mixed in distilled water to obtain a 35 mg/ml solution (referred to as “Mix B”).

Compound B - Zinc gluconate in water solution

| Ingredients | Amount |
|---|---|
| Zinc gluconate (mg) | 350 mg |
| Distilled water | 10 ml |

Device Preparation

0.5 ml of compound A is loaded into chamber A and 4.5 ml of compound B is loaded into chamber B. Upon pressing on the plunger rod, the two compounds mix. The resulting LNE, containing 10 mg ticagrelor and 31.5 mg zinc gluconate is administered orally.

Any of the above protocols or similar variants thereof can be described in various documentation associated with a pharmaceutical product. This documentation can include, without limitation, protocols, statistical analysis plans, investigator brochures, clinical guidelines, medication guides, risk evaluation and mediation programs, prescribing information and other documentation that may be associated with a pharmaceutical product. It is specifically contemplated that such documentation may be physically packaged with a pharmaceutical product according to the present disclosure as a kit, as may be beneficial or as set forth by regulatory authorities.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations are not intended to convey that the claimed subject matter requires features or combinations of features other than those expressly recited in the claims. Accordingly, the scope of this disclosure is intended to include all modifications and variations encompassed within the spirit and scope of the following appended claims.

We claim:

1. A method of treating a subject diagnosed with an autism spectrum disorder, an intellectual disability, epilepsy, an anxiety disorder, a mood disorder, a disorder of social interaction, irritability, aggression, self-injurious behavior, hyperactivity, inattention, Fragile X syndrome, or a subject diagnosed with elevated TNFα, or with elevated inflammatory cytokine markers of neuroinflammation, encompassing administering a liquid lipid nanocarrier emulsion (LNE) formulation comprising ticagrelor, an enantiomer thereof, or a pharmaceutically acceptable salt thereof dissolved in a mixture comprising an oil, a surfactant and a cosurfactant (an oil/surfactant/cosurfactant mixture) and a second agent dissolved in an aqueous solution.

2. The method of claim 1, wherein the LNE formulation comprises the oil/surfactant/cosurfactant mixture and the aqueous solution in a ratio of 1:0.1 to 1:100.

3. The method of claim 1, comprising, before said administering step, providing:
a) a first container containing an oil/surfactant/cosurfactant mixture comprising ticagrelor, an enantiomer thereof, or a pharmaceutically acceptable salt thereof dissolved in a mixture comprising an oil, a surfactant and a cosurfactant; and
b) a second container containing an aqueous solution comprising a magnesium ion containing-compound, a zinc ion containing-compound, L-lysine or a salt thereof, L-arginine or a salt thereof, lecithin, or a combination thereof.

4. The method of claim 1, comprising, before said administering step, providing a dual chamber cartridge, wherein a first chamber of the dual chamber cartridge contains the oil/surfactant/cosurfactant mixture comprising ticagrelor, an enantiomer thereof, or a pharmaceutically acceptable salt thereof dissolved in a mixture comprising an oil, a surfactant and a cosurfactant, and a second chamber of the dual chamber cartridge contains the aqueous solution comprising a magnesium ion containing-compound, a zinc ion containing-compound, L-lysine or a salt thereof, L-arginine or a salt thereof, lecithin, or a combination thereof, wherein the dual chamber cartridge is configured for mixing contents of the first chamber and the second chamber to form the LNE before the administering step.

5. The method of claim 1, comprising administering the LNE formulation orally or parenterally.

6. The method of claim 1, comprising administering 10 to 60 mg/day of the ticagrelor, enantiomer or pharmaceutically acceptable salt thereof.

7. The method of claim 1, comprising administering the LNE formulation in an amount sufficient to achieve a ticagrelor plasma level of about 50 to 1000 ng/ml.

8. The method of claim 7, comprising administering the LNE formulation thereof such that said ticagrelor plasma level is maintained for at least 5 hours.

9. The method of claim 1, comprising administering the LNE formulation orally.

10. The method of claim 1, wherein the subject is diagnosed with an anxiety disorder.

11. The method of claim 1, wherein the subject is diagnosed with irritability, aggression, self-injurious behavior, hyperactivity, or inattention.

12. The method of claim 1, wherein the subject is diagnosed with Fragile X syndrome.

13. The method of claim 1, wherein the subject is diagnosed with elevated TNFα.

14. The method of claim 1, wherein the subject is diagnosed with an elevated inflammatory cytokine marker of neuroinflammation.

15. A method of treating a subject diagnosed with an autism spectrum disorder, an intellectual disability, epilepsy, an anxiety disorder, a mood disorder, a disorder of social interaction, irritability, aggression, self-injurious behavior, hyperactivity, inattention, Fragile X syndrome, or diagnosed with elevated TNFα, or diagnosed with elevated inflammatory cytokine marker of neuroinflammation comprising administering a combination of ticagrelor, an enantiomer thereof, or a pharmaceutically acceptable salt thereof dissolved in a mixture comprising an oil, a surfactant and a cosurfactant (an oil/surfactant/cosurfactant mixture) with a second agent dissolved in an aqueous solution wherein:
a) the combination comprises the oil/surfactant/cosurfactant mixture in a 1:0.1 to 1:100 by volume to the aqueous solution; and
b) the second agent is selected from the group consisting of a magnesium ion containing-compound, a zinc ion containing-compound, L-lysine or a salt thereof, L-arginine or a salt thereof, lecithin, or a combination thereof.

16. The method of claim 15, wherein the second agent is a magnesium ion containing-compound.

17. The method of claim 15, wherein the second agent is a zinc ion containing-compound.

18. The method of claim 15, wherein the second agent is L-lysine or a salt thereof.

19. The method of claim 15, wherein the second agent is L-arginine or a salt thereof.

20. The method of claim 15, wherein the second agent is lecithin.

21. A method of treating a subject diagnosed with an intellectual disability, irritability, aggression, self-injurious behavior, hyperactivity, inattention, Fragile X syndrome, or diagnosed with elevated TNFα, or diagnosed with elevated inflammatory cytokine marker of neuroinflammation comprising administering to the subject a) ticagrelor, an enantiomer thereof, or a pharmaceutically acceptable salt thereof, or ticagrelor, an enantiomer thereof, or a pharmaceutically acceptable salt thereof dissolved in an oil/surfactant/cosurfactant mixture, in combination with b) a second agent dissolved in an aqueous solution wherein:
a) the oil/surfactant/cosurfactant mixture to aqueous solution ratio is 1:0.1 to 1:100; and
b) the second agent is selected from the group consisting of a magnesium ion containing-compound, a zinc ion containing-compound, L-lysine or a salt thereof, L-arginine or a salt thereof, lecithin, or a combination thereof.

22. A liquid lipid nanocarrier emulsion (LNE) formulation comprising ticagrelor, an enantiomer thereof, or a pharmaceutically acceptable salt thereof dissolved in a mixture comprising an oil, a surfactant and a cosurfactant (an oil/surfactant/cosurfactant mixture) and a second agent dissolved in an aqueous solution, wherein the second agent is selected from the group consisting of a magnesium ion containing-compound, a zinc ion containing-compound, L-lysine or a salt thereof, L-arginine or a salt thereof, lecithin, or a combination thereof.

23. The LNE formulation of claim 22, comprising the oil/surfactant/cosurfactant mixture in a 1:0.1 to 1:100 ratio by volume to the aqueous solution.

24. The LNE formulation of claim 22, wherein the oil is selected from a caprylic acid glyceride, a capric acid glyceride, a polyoxylglyceride, an isopropyl myristate, an isopropyl palmitate, a triglyceride, a propylene glycol oil, or glyceryl monooleate.

25. The LNE formulation of claim 22, wherein the surfactant is selected from a polysorbate, a polyoxyethylene castor oil derivatives, a caprylocaproyl macrogol glyceride, a sorbitan, a polyoxyethylene alkyl ether, a polyoxyethylene sorbitan fatty acid, a polyoxyethylene-polyoxypropylene copolymer, an alkyl sulfonate, an alkyl sulfate a quaternary ammonium salt, a fatty alcohol, a glycerylester, a fatty acid ester, or a polyoxyethylene.

26. The LNE formulation of claim 22, wherein the cosurfactant is selected from a tetraglycol, a propylene glycol, a diethylene glycol monoethyl ether, a polyethylene glycol, hexanol, pentanol, or octanol.

27. The LNE formulation of claim 22, wherein the LNE formulation comprises 5% to 50% by weight of oil, 5% to 80% by weight of surfactant and 5% to 80% by weight of the cosurfactant based on the total weight of the oil/surfactant/cosurfactant mixture.

28. The LNE formulation of claim 22, wherein the LNE formulation comprises 10% to 20% by weight of oil, 20% to 60% by weight of surfactant and 15% to 60% by weight of the cosurfactant based on the total weight of the oil/surfactant/cosurfactant mixture.

29. The LNE formulation of claim 22, wherein the LNE formulation comprises a weight ratio of 15:1 to 3:1 (w/w) oil/surfactant/cosurfactant mixture to ticagrelor.

30. The LNE formulation of claim 22, comprising 10-90 mg of the ticagrelor, enantiomer thereof, or pharmaceutically acceptable salt thereof.

31. The LNE formulation of claim 22, wherein the weight ratio of the oil:surfactant ranges from 1:0.5 to 1:80.

32. The LNE formulation of claim 22, wherein the weight ratio of the oil:cosurfactant ranges from 1:0.4 to 1:50.

33. The LNE formulation of claim 22, wherein the weight percentage of the ticagrelor, enantiomer thereof, or pharmaceutically acceptable salt thereof to the oil/surfactant/cosurfactant mixture ranges from 1:3 to 1:15 respectively.

34. The LNE formulation of claim 22, wherein the LNE formulation comprises the second agent in a concentration of 1 to 300 mg/ml.

35. The LNE formulation of claim 22, wherein the molar ratio of the ticagrelor, enantiomer thereof, or pharmaceutically acceptable salt thereof to the second agent in the LNE formulation ranges from 1:0.1 to 1:100 (mol/mol).
